(19) United States
(12) Plant Patent Application Publication
(10) Pub. No.: US 2003/0106112 P1
(43) Pub. Date: Jun. 5, 2003

(54) Shrub rose plant named JACdreco

(75) Inventor: Keith W. Zary, Thousand Oaks, CA (US)

Correspondence Address: KLARQUIST SPARKMAN, LLP, 121 SW SALMON STREET, SUITE 1600, PORTLAND, OR, 97204, US

(73) Assignee: Jackson & Perkins Wholesale, Inc.

(21) Appl. No.: 10/000,259

(22) Filed: Nov. 30, 2001

(51) Int. Cl. A01H 5/00

(52) U.S. Cl. PLT/102

(57) ABSTRACT

Shrub rose plant having large pyramidal flower clusters; small, dark green glossy foliage; resistance to major fungal diseases; novel purplish pink flower color; many small prickles on strong shoots, none on side shoots; and ability to root and grow well from softwood cuttings.

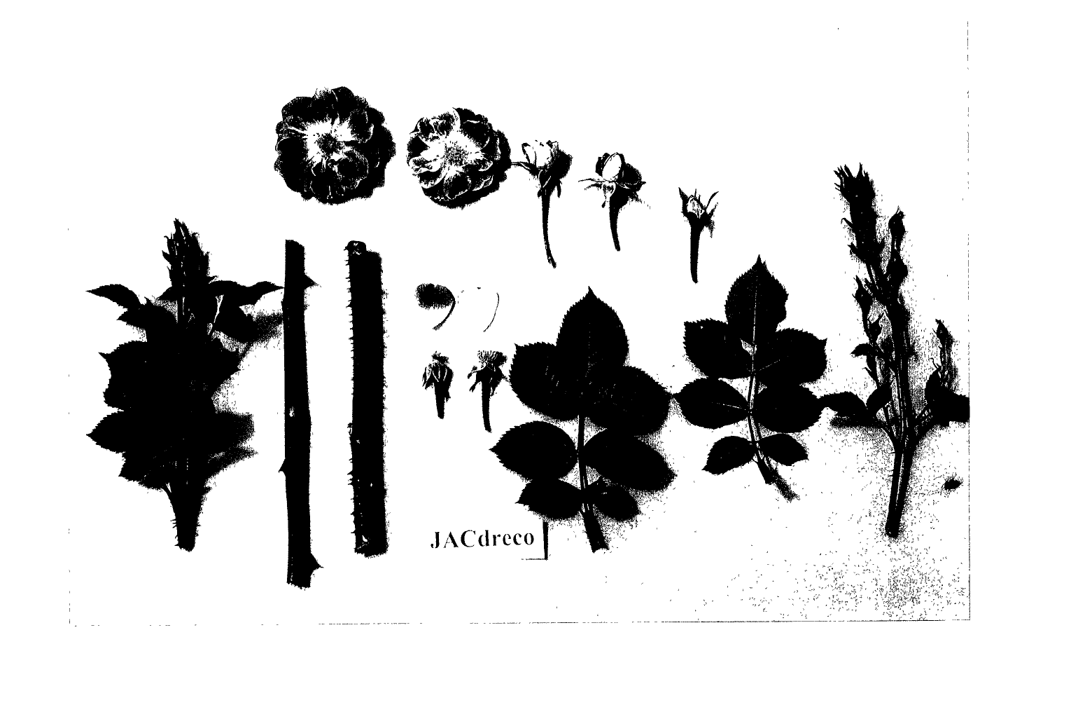

### SUMMARY OF THE INVENTION

The present invention relates to a new and distinct variety of rose plant of the shrub class which was originated by me by crossing the variety INTERlav (U.S. Plant Pat. No. 5,916) with the variety MACminmo (U.S. Plant Pat. No. 7,319).

### BACKGROUND OF THE INVENTION

The primary objective of this breeding was to produce a new rose variety having the large flower clusters, small, dark green foliage and vigorous growth of the female parent combined with the glossy foliage and disease resistance of the male parent.

### BRIEF SUMMARY OF THE INVENTION

The objective was substantially achieved, along with other desirable improvements, as evidenced by the following unique combination of characteristics that are outstanding in the new variety and that distinguish it from its parents, as well as from all other varieties of which I am aware:

1. Large, pyramidal flower clusters;

2. Small, dark green, glossy foliage;

3. Resistance to major fungal diseases;

4. Novel purplish pink flower color;

5. Many small prickles on strong shoots, none on side shoots; and

6. Ability to root and grow well from softwood cuttings.

Asexual reproduction of this new variety by rooting cuttings, as performed at Wasco, Calif., shows that the foregoing and all other characteristics and distinctions come true to form and are established and transmitted through succeeding propagations.

### COMPARISON WITH PARENT

Differs from female in flower color, leaf gloss and number of small prickles. Also differs from male in flower color and in plant habit.

### COMPARISON WITH SIMILAR VARIETY

There are no roses of which I am aware that are similar to JACdreco.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with The Royal Horticultural Society (London) colour chart, except where ordinary dictionary significance of color is indicated.

Parentage:

Seed parent.—INTERlav (U.S. Pat. No. 5,916).

Pollen parent.—MACminmo (U.S. Pat. No. 7,319).

Classification:

Botanical.—Rosa hybrida.

Commercial.—Shrub.

### FLOWER

Observations made from plants of 2 years of age grown in a garden environment at Somis, Calif., January, 2000- August, 2001.

Blooming Habit: Continuous.

Bud:

Size.—12-14 mm long when the petals start to unfurl.

Form.—The bud form is pointed ovoid.

Color.—When sepals first divide, bud color is Red-Purple Group 60B with Red-Purple Group 62D on petal edges and Yellow Group 8C at the base. When half blown, the upper sides of the petals are close to Red-Purple Group 58B and Yellow Group 5D at the base; and the lower sides of the petals are White Group 155D with Red-Purple Group 62D edges.

Sepals.—Color: Upper surface: Green Group 141D. Lower surface: Greyed-Purple Group 183D with Yellow-Green 144B on margins. Length: 16-18 mm. Width: 5-6 mm. Shape: Subulate. Surface texture: Upper: Hoary. Lower: Smooth. Margins: Glandular. There are three lightly appendaged sepals. There are two unappendaged sepals with hairy, glandular edges.

Receptacle.—Color: Green Group 143B. Shape: Funnel. Size: Medium, about 5 mm wide ×7 mm long. Surface: Hairy, hoary.

Peduncle.—Length: Short, averaging about 20-24 mm. Surface: Glandular, many. Color: Yellow-Green Group 146B with anthocyanin of Greyed-Orange Group 176B. Strength: Stiff, erect.

Bloom:

Size.—Small. Average open size is 48-52 mm.

Borne.—In pyramidal clusters of 10-50 blooms.

Stems.—Strength: Strong. Average length is about 30-50 cm.

Form.—When first open: Slightly cupped to flat.

Permanence.—Retains its form to the end.

Petalage.—Number of petals under normal conditions: 10-15.

Color.—The upper sides of the petals are Red-Purple Group 58A in center and Red-Purple Group 58B on edges and Yellow Group 5D at base. The reverse sides of the petals are White Group 15D with Red-Purple Group 62D edges. The major color on the upper side is Red-Purple Group 58B.

Discoloration.—The general tonality at the end of the first day: Red-Purple Group 58B. At the end of the third day: Red-Purple Group 63B.

Fragrance.—Slight. Character of fragrance: Pungent like that of a spinosissema rose.

Petals:

Texture.—Thin.

Shape.—Obcordate.

Margin.—Entire.

Apex shape.—Obcordate.

Base shape.—Cuneate.

Form.—Flat.

Arrangement.—Imbricated. Petaloids in the center are few (1-2). Petaloid color: Same as petals.

Persistence.—Petals drop off cleanly before drying.

Lastingness.—On the plant: Short (about 4-5 days). As a cut flower: Short (about 4-5 days).

Reproductive Parts:

Stamens.—Number: 80-100. Length: 5-7 mm. Anthers: Size: 1 mm. Color: Orange Group 26B. Arrangement: Regular around styles. Filaments: Color: Yellow-Orange Group 15C. Length: 4-6 mm.

Pollen.—Color: Greyed-Orange Group 163C.

Pistils.—Number: 35-40. Styles: Color: Greyed-Yellow Group 162D with variegation of Red Group 53C. Length: 3-4 mm. Stigmas: Color: Greyed-Orange Group 163C.

Hips.—Color: Immature: Green Group 146B. Shape: Apple. Size: 10 mm wide ×8 mm long.

### PLANT

Form.—Bush.

Growth.—Very vigorous. upright and branching.

Mature plant.—Height is 120 cm, Width is 80 cm.

Foliage: Number of leaflets on normal mid-stem leaves: 5 or 7.

Size.—Small (10 cm long ×6 cm wide).

Quantity.—Abundant.

Color.—New foliage: Upper side: Yellow-Green Group 146A Lower side: Greyed-Red Group 178B. Old foliage: Upper side: Green Group 139A. Lower side: Green Group 137C.

Leaflets:

Size.—Small (1.5 cm long ×1 cm wide).

Shape.—Oval.

Base shape.—Rounded.

Apex shape.—Acuminate.

Texture.—Glossy, smooth.

Edge.—Serrated.

Serrations.—Single, small.

Petiole color.—Green Group 137B.

Petiole rachis.—Color: Yellow-Green Group 146C.

Petiole underside.—Rough with glands.

Stipules.—Short (about 10-12 mm.)

Disease resistance.—Resistant to mildew, blackspot and rust under normal growing conditions.

Pest persistance.—Not observed.

Wood:

New wood.—Color: Yellow-Green Group 146C except where exposed to bright sunlight, then it is Greyed-Red Group 178A. Bark: Smooth.

Old wood.—Color: Green Group 137C. Bark: Smooth.

Prickles:

Quantity.—On main canes from base: Many to ordinary. On laterals from main canes: Ordinary.

Form.—Hooked downward. Length: 6-7 mm.

Color when young.—Greyed-Purple Group 183B.

Color when mature.—Greyed-Orange Group 177C.

Small Prickles:

Quantity.—On main stalks: Many. On laterals: None.

Color.—Young: Greyed-Purple Group 183B; Mature: Greyed-Orange Group 177C.

Winter Hardiness: Hardy to Zone 5.

I claim:

1. A new and distinct variety of rose plant of the shrub class, substantially as herein shown and described, characterized particularly by its large pyramidal flower clusters; small, dark green glossy foliage; resistance to major fungal diseases; novel purplish pink flower color; many small prickles on strong shoots, none on side shoots; and ability to root and grow well from softwood cuttings.
